SEQUENCE LISTING (7 sequences)

SEQ ID NO: 1 - DNA, 2196 bp, human
  CDS (13)..(1983)
aattccggagccatggtgaacgaagccagaggaaacagcagcctcaacccctgcttggagggcagtgccagcagtggcagtgagagctccaaagatagttcgagatgttccaccccgggcctggaccctgagcggcatgagagactccgggagaagatgaggcggcgattggaatctggtgacaagtggttctccctggaattcttccctcctcgaactgctgagggagctgtcaatctcatctcaaggtttgaccggatggcagcaggtggccccctctacatagacgtgacctggcacccagcaggtgaccctggctcagacaaggagacctcctccatgatgatcgccagcaccgccgtgaactactgtggcctggagaccatcctgcacatgacctgctgccgtcagcgcctggaggagatcacgggccatctgcacaaagctaagcagctgggcctgaagaacatcatggcgctgcggggagacccaataggtgaccagtgggaagaggaggagggaggcttcaactacgcagtggacctggtgaagcacatccgaagtgagtttggtgactactttgacatctgtgtggcaggttaccccaaaggccaccccgaagcagggagctttgaggctgacctgaagcacttgaaggagaaggtgtctgcgggagccgatttcatcatcacgcagcttttctttgaggctgacacattcttccgctttgtgaaggcatgcaccgacatgggcatcacttgccccatcgtccccgggatctttcccatccagggctaccactcccttcggcagcttgtgaagctgtccaagctggaggtgccacaggagatcaaggacgtgattgagccaatcaaagacaacgatgctgccatccgcaactatggcatcgagctggccgtgagcctgtgccaggagcttctggccagtggcttggtgccaggcctccacttctacaccctcaaccgcgagatggctaccacagaggtgctgaagcgcctggggatgtggactgaggaccccaggcgtcccctaccctgggctctcagtgcccaccccaagcgccgagaggaagatgtacgtcccatcttctgggcctccagaccaaagagttacatctaccgtacccaggagtgggacgagttccctaacggccgctggggcaattcctcttcccctgcctttggggagctgaaggactactacctcttctacctgaagagcaagtcccccaaggaggagctgctgaagatgtggggggaggagctgaccagtgaagcaagtgtctttgaagtctttgttctttacctctcgggagaaccaaaccggaatggtcacaaagtgacttgcctgccctggaacgatgagcccctggcggctgagaccagcctgctgaaggaggagctgctgcgggtgaaccgccagggcatcctcaccatcaactcacagcccaacatcaacgggaagccgtcctccgaccccatcgtgggctggggccccagcgggggctatgtcttccagaaggcctacttagagtttttcacttcccgcgagacagcggaagcacttctgcaagtgctgaagaagtacgagctccgggttaattaccaccttgtcaatgtgaagggtgaaaacatcaccaatgcccctgaactgcagccgaatgctgtcacttggggcatcttccctgggcgagagatcatccagcccaccgtagtggatcccgtcagcttcatgttctggaaggacgaggcctttgccctgtggattgagcggtggggaaagctgtatgaggaggagtccccgtcccgcaccatcatccagtacatccacgacaactacttcctggtcaacctggtggacaatgacttcccactggacaactgcctctggcaggtggtggaagacacattggagcttctcaacaggcccacccagaatgcgagagaaacggaggctccatgaccctgcgtcctgacgccctgcgttggagccactcctgtcccgccttcctcctccacagtgctgcttctcttgggaactccactctccttcgtgtctctcccaccccggcctccactcccccacctgacaatggcagctagactggagtgaggcttccaggctcttcctggacctgagtcggccccacatgggaacctagtactctctgctcta

SEQ ID NO: 2 - protein, 656 aa, human
MVNEARGNSSLNPCLEGSASSGSESSKDSSRCSTPGLDPERHERLREKMRRRLESGDKWFSLEFFPPRTAEGAVNLISRFDRMAAGGPLYIDVTWHPAGDPGSDKETSSMMIASTAVNYCGLETILHMTCCRQRLEEITGHLHKAKQLGLKNIMALRGDPIGDQWEEEEGGFNYAVDLVKHIRSEFGDYFDICVAGYPKGHPEAGSFEADLKHLKEKVSAGADFIITQLFFEADTFFRFVKACTDMGITCPIVPGIFPIQGYHSLRQLVKLSKLEVPQEIKDVIEPIKDNDAAIRNYGIELAVSLCQELLASGLVPGLHFYTLNREMATTEVLKRLGMWTEDPRRPLPWALSAHPKRREEDVRPIFWASRPKSYIYRTQEWDEFPNGRWGNSSSPAFGELKDYYLFYLKSKSPKEELLKMWGEELTSEASVFEVFVLYLSGEPNRNGHKVTCLPWNDEPLAAETSLLKEELLRVNRQGILTINSQPNINGKPSSDPIVGWGPSGGYVFQKAYLEFFTSRETAEALLQVLKKYELRVNYHLVNVKGENITNAPELQPNAVTWGIFPGREIIQPTVVDPVSFMFWKDEAFALWIERWGKLYEEESPSRTIIQYIHDNYFLVNLVDNDFPLDNCLWQVVEDTLELLNRPTQNARETEAP

SEQ ID NO: 3 - DNA, 2256 bp, human
aagctttcttctgcagaggccacctagtcatgtttctcaccctgcacctcagcctccccactccatctctcaatcatgcctagggtttggaggaaggcatttgattctgttctggagcacagcagaagaattgacatcctcaaaattaaaactcccttgcctgcacccctccctcagatatctgattcttaatgtctagaaaggaatctgtaaattgttccccaaatattcctaagctccatcccctagccacaccagaagacacccccaaacaggcacatctttttaattcccagcttcctctgttttggagaggtcctcagcatgcctctttatgcccctcccttagctcttgccaggatatcagagggtgactggggcacagccaggaggaccccctccccaacacccccaacccttccacctttggaagtctccccacccagctccccagttccccagttccacttcttctagattggaggtcccaggaagagagcagaggggcacccctacccactggttagcccacgccattctgaggacccagctgcacccctaccacagcacctctggcccaggctgggctggggggctggggaggcagagctgcgaagaggggagatgtggggtggactcccttccctcctcctccccctctccattccaactcccaaattgggggccgggccaggcagctctgattggctggggcacgggcggccggctccccctctccgaggggcagggttcctccctgctctccatcaggacagtataaaaggggcccgggccagtcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacagtaagtcccaaacttttgggagtgcaaggatactctatatcgcgccttgcgcttggtcccgggggccgcggcttaaaacgagacgtggatgatccggagactcgggaatggaagggagatgatgagggctcttcctcggcgccctgagacaggagggagctcaccctggggcgaggttggggttgaacgcgccccgggagcgggaggtgagggtggagcgcggcgtgagttggtgcaagagagaatcccgagcgcgcaaccggggaagtggggatcttgggtgcagagtgaggaaagcacgtcgaagatgggatgggggcgccgagcggggcatttgaagcccaagatgtagaagcaatcaggaaggccgtgggatgattcataaggaaagattgccctctctgcgggctagagtgttgctggggccgtgggggtgctgggcagccgcggagggggtgcggagcgtgggcgggtggaggatgagaaactttggcgcggactcggcggggcggggtccttgcgccccctgctgaccgatgctgagcactgcgtctcccggtccaacgcttactggggcaggagccggagcgggaagacccgggttattgctgggtgcggacccccacctgctagatctggaaagtaaagccagggatggggcagcccaagcctcttaaagaggtagtcgggccggtgaggtcggccccgccccggccccattgcttagcgttgcccgacacctagtggccgtctggggagccgctagcgcggtgggagtggttagctaacttctggactatttgcggactttttggttctttggctaaaagtgacctggaggcattggctggctttggggactggggatggcccgagagcggcttttaagttgtctaggtgctggaggttagggtgtctcctaatttaggtacatttcaagtcttgggggggcctcccttccaatcagccgctcccattctcctagccccgcccccgccaccccacctgcccagggaatgggggcgggatgagggctggacctcccttctctcctccctcgccctcctcctgtctctaccacgcaagccactccccacgagcctgccctcccgatggggcccctcctattctccccccgccctccccctctcaccctgtggtttttatttcacttggcttcagcgccaatgggctgaggttggagttggaagccaccgcggactaaagctt

SEQ ID NO: 4 - DNA, 23 bp, human
tgaaggagaaggtgtctgcggga

SEQ ID NO: 5 - DNA, 20 bp, human
aggacggtgcggtgagagtg

SEQ ID NO: 6 - DNA, 30 bp, human
taacttctggactatttgcggactttttgg

SEQ ID NO: 7 - DNA, 20 bp, human
gtccagccctcatcctggcc

(19) United States
(12) Patent Application Publication
(10) Pub. No.: US 2016/0215339 A1
(43) Pub. Date: Jul. 28, 2016

(54) SUSCEPTIBILITY TO BONE DAMAGE

(71) Applicant: BodySync, Inc., Redondo Beach, CA (US)

(72) Inventors: Andreas Gerardus Uitterlinden, Poortugal (NL); Joyce Berdina Josepha van Meurs, Delft (NL)

(21) Appl. No.: 13/890,897

(22) Filed: May 9, 2013

Related U.S. Application Data
(63) Continuation of application No. 13/170,296, filed on Jun. 28, 2011, now abandoned.
Application No. 13/170,296 is a continuation of application No. 12/826,142, filed on Jun. 29, 2010, now abandoned.
Application No. 12/826,142 is a continuation of application No. 12/147,362, filed on Jun. 26, 2008, now abandoned.
Application No. 12/147,362 is a continuation of application No. 10/270,714, filed on Oct. 11, 2002, now abandoned.
(60) Provisional application No. 60/328,929, filed on Oct. 11, 2001.

(51) Int. Cl. C12Q 1/68; A61K 31/519

(52) U.S. Cl. CPC C12Q 1/6883; A61K 31/519; C12Q 2600/118; C12Q 2600/172; C12Q 2600/156

(57) ABSTRACT

In one aspect, the present invention provides methods for determining susceptibility to bone damage in a subject. In some embodiments, the methods comprise screening for polymorphisms in the MTHFR and collagen Iα1 genes that are associated with susceptibility to bone damage. In some embodiments, the methods comprise screening for elevated levels of homocysteine in a subject, wherein elevated levels of homocysteine are associated with an increased risk of bone damage. The methods of the invention may be used in predicting the response of a patient to treatment. Also provided are methods for prevention or reducing the risk of bone damage in a subject.

### CROSS-REFERENCE TO RELATED APPLICATION

This application claims the benefit of U.S. Provisional Application No. 60/328,929, filed Oct. 11, 2001, under 35 U.S.C. §119.

### FIELD OF THE INVENTION

The present invention relates to methods for determining susceptibility to bone damage in a subject, methods for predicting the response of a subject to treatment, and methods for prevention of bone damage in a subject.

### BACKGROUND OF THE INVENTION

Osteoporosis is a common disease characterized by reduced bone mineral density (BMD), deterioration of bone micro-architecture and increased risk of bone damage, such as fracture. It is a major public health problem, which affects quality of life and increases costs to health care providers. In European populations, one in three women and one in twelve men over the age of fifty is at risk. The disease affects 25 million people in the USA, where the incidence of disease is 25% higher than it is in the UK, and a further 50 million people in Japan and Europe combined. It is estimated that by the middle of the next century the number of osteoporosis sufferers will double in the West, but may increase six-fold in Asia and South America. Fracture is the most serious endpoint of osteoporosis, particularly fracture of the hip which affects up to 1.7 million people worldwide each year. It is estimated that by the year 2050, the number of hip fractures worldwide will increase to over 6 million, as life expectancy and age of the population increase.

There are multiple factors that contribute to the development of osteoporosis. Low BMD is an important risk factor for fractures, the clinically most relevant feature of osteoporosis.

There is evidence from twin and family studies indicating that genetic factors play a major role in the pathogenesis of osteoporosis (Smith et al. (1973) J. Clin. Invest. 52:2800-8; Pocock et al. (1987) J. Clin. Invest. 8-:706-10; Evans et al. (1988) Ann. Intern. Med. 109:870-3; Seeman et al. (1989) N. Engl. J. Med. 320:554-8). Osteoporosis is a polygenetic trait with variants of several genes underlying the susceptibility to the disease. An important candidate gene is the collagen type Iα1 (COLIα1) gene, which encodes the α1-chain of the most abundant protein of bone matrix: collagen type I. A functional regulatory polymorphism in COLIα1 has previously been shown to be associated with differences in BMD and risk of osteoporotic fracture (Grant et al. (1996) Nature Genet. 14:303-5; Uitterlinden et al. (1998) N. Engl. J. Med. 338:1017-1021; Mann et al. (2001) J. Clin. Invest. 107:899-907).

Another metabolic pathway that may be involved in osteoporosis is homocysteine metabolism. A rare autosomal recessive disease, homocystinuria, is characterized by highly elevated levels of plasma homocysteine and is accompanied by several clinical manifestations including osteoporosis (Harpey et al. (1981) J. Pediatr. 98:275-8; Mudd et al. (1985) Am J. Hum. Genet. 37:1-31). The underlying pathobiological mechanism for the occurrence of early osteoporosis in homocystinuria patients is not completely understood. However, in vivo and in vitro studies support disturbed cross-linking of collagen type I in bone as a possible explanation (McKusick (1966) in Heritable disorders of connective tissue, p. 155; Harris & Sjoerdsma (1966) Lancet 2:707-11; Kang & Trestad (1973) J. Clin. Invest. 52:2571-8; Jackson (1973) Clin. Chim. Acta 45:215-7; Lubec et al. (1996) Biochim. Biophys. Acta 1315:159-62). Therefore, it is possible that homocysteine and collagen type I interact to determine bone quality.

In the general population, a mildly elevated level of plasma homocysteine is a common condition. A key enzyme in homocysteine metabolism is methyltetrahydrofolate reductase (MTHFR). A commonly occurring variant of this enzyme (Kang et al. (1991) Am. J. Hum. Genet. 48:536-45; Frosst et al. (1995) Nat. Genet. 10:111-3), the 222-Val variant, results in a reduced enzymatic activity. In some populations, this MTHFR variant has been associated with mildly elevated homocysteine levels (Christensen et al. (1997) Arterioscler. Throm. Vasc. Biol. 17:569-73; Chango et al. (2000) Br. J. Nutr. 83:593-6). Recently, this variant was also found to be associated with low BMD (Miyao et al. (2000) Calcif. Tissue Int. 66:190-194). However, this variant cannot be used as an increased risk for bone damage itself because it has previously been shown that bone damage in osteoporosis can be independent of BMD.

Strategies for the prevention of this disease in those at risk include development of bone density in early adulthood, and minimization of bone loss in later life. For example, changes in lifestyle, nutrition and hormonal factors have been shown to affect bone loss. Treatment of osteoporosis is unsatisfactory. In particular, once bone damage has occurred as a result of osteoporosis, there is little a physician can do other than let the bone heal. In the elderly, this may be a slow and painful process. Diagnosis of those at risk of developing bone damage would allow more effective preventative measures. Accordingly, there is a need for methods for diagnosing and treating those at risk for developing bone damage.

### SUMMARY OF THE INVENTION

In a first aspect, the present invention provides a method for determining susceptibility to bone damage in a living subject, based upon screening for polymorphisms in the collagen Iα1 and/or methyltetrahydrofolate reductase (MTHFR) genes, wherein the polymorphism(s) is/are associated with susceptibility to bone damage. In some embodiments of the method, the polymorphism in the collagen Iα1 gene is the Sp1 polymorphism, and the polymorphism in the MTHFR gene is the C677T polymorphism. Thus, in some embodiments, the method comprises determining the presence of the T allele of the C677T polymorphism of the MTHFR gene and/or the s allele of the SpI polymorphism of the collagen Iα1 gene in a living subject, wherein the presence of at least one copy of the T allele of the C677T polymorphism of the MTHFR gene and at least one copy of the s allele of the SpI polymorphism of the collagen Iα1 gene in the subject is indicative of an increased susceptibility to bone damage. The method of this aspect of the present invention may also optionally include screening for other polymorphisms in the MTHFR gene and/or the collagen Iα1 gene that are useful in determining risk of bone damage.

In a second aspect, the invention provides a method for determining risk of bone damage in a living subject, comprising measuring the level of serum homocysteine in a living subject, wherein the presence of an elevated level of serum homocysteine, compared to the level of serum homocysteine in a reference population is indicative of an increased risk of bone damage. The reference population is typically a population of living organisms of the same species and sex as the living subject whose risk of bone damage is being determined. In some embodiments, a level of serum homocysteine that is greater than about 20 μmol/l is indicative of an increased susceptibility to bone damage.

In a third aspect, the invention provides a method for preventing or reducing bone damage in a subject. In some embodiments of the method, the subject has been diagnosed as being at risk of bone damage, for example by determining the presence of alleles of the MTHFR and collagen Iα1 loci that are associated with an increased risk of bone damage, or by determining the presence of an elevated level of plasma homocysteine that is associated with an increased risk of bone damage. The method for preventing or reducing bone damage in a subject includes any means of reducing the risk of bone damage in a subject. In some embodiments, the method comprises administering an agent for reducing the levels of homocysteine in plasma. The agent may be, for example, folic acid or folate.

In a fourth aspect, the invention provides a method for predicting the response of a subject to treatment to reduce the risk of bone damage, comprising determining which allele(s) of polymorphism of MTHFR and/or collagen Iα1 are present. Some embodiments provide a method for predicting the response of a subject to treatment with folic acid to reduce the risk of bone damage, wherein the method comprises the steps of: (1) determining which allele(s) of polymorphisms in an MTHFR gene are present in a living subject; and (2) predicting the response of the subject to treatment with folic acid, wherein the absence of an allele of a polymorphism in MTHFR that is associated with an increased risk of bone damage is indicative that treatment of the subject with folic acid is unlikely to reduce the risk of bone damage. The polymorphism in the MTHFR gene may be the C677T polymorphism. Some embodiments provide a method for predicting the response of a living subject to treatment with folic acid to reduce the risk of bone damage, wherein the method comprises the steps of: (1) determining the presence of an elevated level of serum homocysteine in a subject, wherein the presence of the elevated level of serum homocysteine compared to the level of serum homocysteine in a reference population is indicative of an increased risk of bone damage; and (2) predicting the response of the subject to treatment with folic acid, wherein the presence of an elevated level of serum homocysteine in the subject is indicative that treatment of the subject with folic acid is likely to reduce the risk of bone damage. The reference population is typically a population of living organisms of the same species and sex as the living subject whose risk of bone damage is being determined.

In a fifth aspect, the invention provides a kit for determining which allele(s) of one or more polymorphism(s) of an MTHFR gene and/or collagen Iα1 gene are present in a living subject.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows the cumulative fracture incidence in 428 women with baseline homocysteine serum levels in the upper age specific quartile and in all other women (quartiles 1, 2, and 3). A=highest age-specific quartile; B=all other women.

FIG. 2 shows the cumulative fracture risk according to combined genotype for COLIA1and MTHFR for all women. A=MTHFR+COLIA1; B=COLIA1; C=MTHFR; D=Reference.

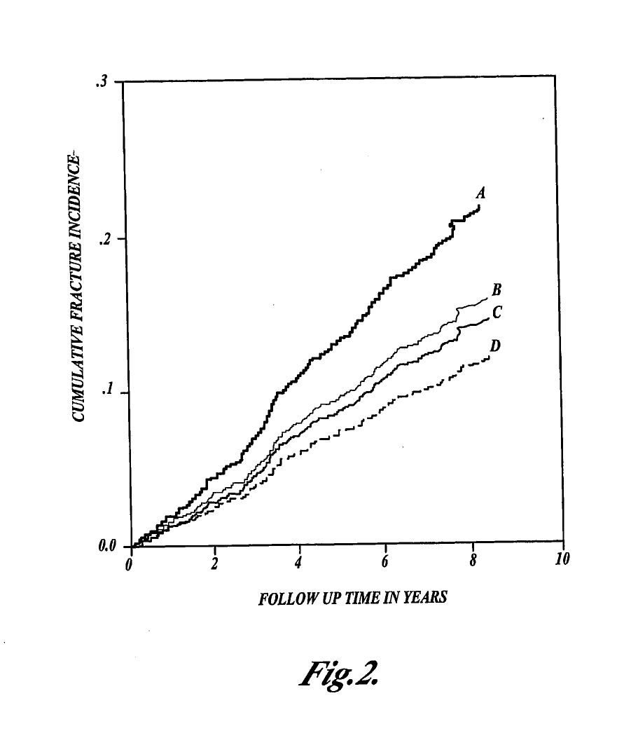

FIG. 3 shows the cumulative fracture risk according to combined genotype for COLIA1 and MTHFR for women older then 66 years at baseline. A=MTHFR+COLIA1; B=COLIA1; C=MTHFR; D=Reference.

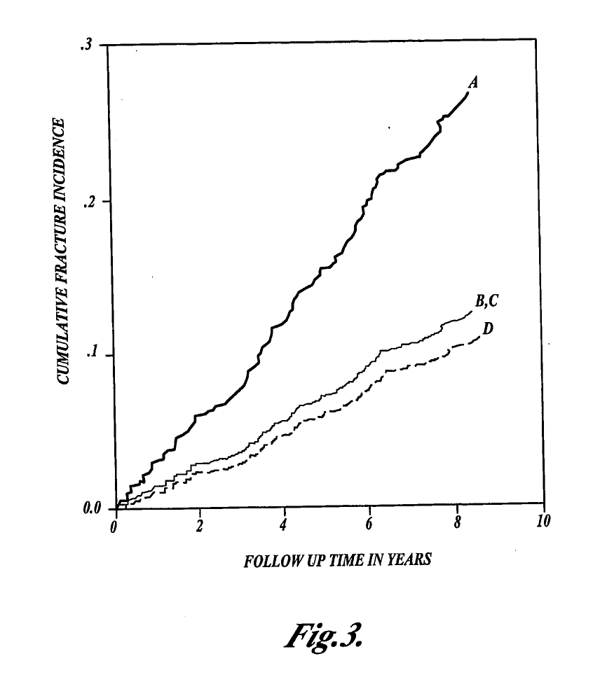

### DETAILED DESCRIPTION OF THE PREFERRED EMBODIMENT

As used herein, the following terms have the meanings defined below:

As used here, the term “MTHFR gene” refers to a gene coding for methyltetrahydrofolate reductase. An exemplary sequence of an MTHFR gene is set forth in SEQ ID NO:1. The amino acid sequence of the protein encoded by the MTHFR gene set forth in SEQ ID NO:1 is set forth in SEQ ID NO:2. The term “MTHFR gene” includes other MTHFR genes that are at least 95% or at least 99% identical to the sequence of SEQ ID NO:1. Sequence identity can be determined, for example, by using the program of Altschul et al. (1997) (Nucleic Acids Res. 25:3389-3402), as embodied in a BLAST program available, for example, at http://www.ncbi.nlm.nih.gov/BLAST/, using default parameters.

There is an inherited polymorphism at position 677 of the nucleotide sequence of the MTHFR gene (see Kang et al. (1991) Am. J. Hum. Genet. 48:536-45; Frosst et al. (1995) Nat. Genet. 10:111-3). This polymorphism consists of a substitution of a C residue at position 677 of the sequence provided in SEQ ID NO:1) with a T residue. The substitution in the nucleotide sequence converts an alanine at codon 222 (herein referred to as “222-Ala”) in the protein sequence provided in SEQ ID NO:2 to a valine (herein referred to as “222-Val” or “222-Val variant”). The genetic polymorphism is denoted “C677T”, where the number refers to the position of the polymorphisms with respect to the nucleotide sequence; the “C” is the nucleotide present in the reference or wild type sequence; and the “T” is the nucleotide residue present at that position in the variant sequence. Thus, the term “T allele” refers to the presence of the T nucleotide at the C677T polymorphic site. Similarly, the amino acid polymorphism is denoted “Ala222Val”. For the purposes of the present invention, determination of which allele is present in a particular nucleotide sequence or protein sequence may be referred to as determining the genotype or phenotype, respectively, of a subject.

As used herein, the term “COLIα1 gene” refers to a gene coding for an α1 chain of collagen type 1. An exemplary sequence of a gene coding for an α1 chain of collagen type 1 is set forth in SEQ ID NO:3. The term “COLIα1 gene” includes other COLIα1 genes that are at least 95% or at least 99% identical to the sequence of SEQ ID NO:3. Sequence identity can be determined, for example, by using the program of Altschul et al. (1997) (Nucleic Acids Res. 25:3389-3402), as embodied in a BLAST program available, for example, at http://www.ncbi.nlm.nih.gov/BLAST/, using default parameters.

A naturally-occurring polymorphism occurs in intron 1 of the COLIα1 gene at position 2046 of the sequence provided in SEQ ID NO:3, located 437 nucleotides 5′ to the start of exon 2 (G-437/in1T). The polymorphism lies in the Sp1 transcription factor binding site and consists of a substitution of the G nucleotide at position 2046 with a T nucleotide. The two different alleles are denoted S/s, where s indicates the presence of a T nucleotide (or T allele) at the polymorphic site. Herein, the polymorphism is referred to as the “Sp1 polymorphism”. The term “collagen Iα1 risk allele ” refers to the presence of the T nucleotide at the polymorphic site, i.e., the s allele at the Sp1 site.

As used herein, the term “bone damage” refers to any form of structural damage including fractures, breaks, or chips. The term may also include biological degradation or deterioration of bone. Typically, the term bone damage does not include low bone mineral density. This is in line with the finding that risk of bone damage is independent of bone mineral density. Fracture may be defined as the clinically most important endpoint, and thus the method of the first aspect of the invention preferably relates to a method for determining risk of fracture. Although such bone damage will usually be the result of osteoporosis, it is irrelevant for the purposes of the present invention whether a subject has first been diagnosed as having osteoporosis.

As used herein, the terms “risk of bone damage” and “susceptibility to bone damage” are used interchangeably.

The first aspect of the invention provides a method for determining susceptibility to bone damage in a subject, comprising determining which alleles of polymorphisms in collagen Iα1 and MTHFR are present. Thus, the present invention enables the identification of those individuals susceptible to bone damage, and the development of therapeutic or preventative measures. For example, those at risk may avoid damage by modifying their lifestyle and implementing bone strengthening measures, such as regular exercise and a healthy diet, or by taking medicaments which reduce the risk of damage. In some embodiments, the method includes the steps of: (1) determining that an allele of at least one polymorphism in a collagen Iα1 gene and/or an MTHFR gene is associated with an increased or a decreased susceptibility to bone damage; (2) determining which one or more alleles associated with an increased or a decreased susceptibility to bone damage is present in a subject; and (3) determining whether the one or more alleles present in the subject are associated with an increased or with a decreased susceptibility to bone damage. The determination that an allele of a polymorphism in a collagen Iα1 gene and/or an MTHFR gene is associated with an increased or a decreased susceptibility to bone damage may be performed using standard statistical analyses in a population of subjects, as described, for example, in EXAMPLE 2. An exemplary method for determining the presence of one or more alleles associated with an increased or a decreased susceptibility to bone damage is present in a subject is described in detail below and in EXAMPLE 2.

Exemplary polymorphisms are the C677T polymorphism of MTHFR and the Sp1 polymorphism of collagen Iα1 are, respectively. The MTHFR gene comprises an inherited polymorphism at position 677 of the nucleotide sequence (Frosst et al. (1995) Nat. Genet. 10:111-3). The sequence of MTHFR and methodology on how to identify the C677T polymorphisms has been previously described (Goyette et al. (1994) Nature Genetics 7:195-200; Goyette et al. (1998) Mammalian Genome 9:652-656).

Collagen Iα1 (17q22) has a G to T polymorphism in the gene at 437 nucleotides before exon 2 in intron 1. The polymorphism, denoted G-437/in1T, lies in the Sp1 transcription factor binding site, and can be detected by MscI restriction enzyme digestion, if amplified by appropriate mis-match primers, such as those described below. Thus, if the T allele is present, a mis-match is introduced which introduces an MscI restriction site. The alleles are denoted S/s, where s indicates the presence of a T allele at the polymorphic site.

The present invention is based upon the surprising observation of a correlation between the presence of the T allele of the MTHFR and susceptibility to/or risk of bone damage, such as fracture in those subjects having the collagen Iα1 risk allele (i.e., the T nucleotide, or s allele at the Sp1 site). A subject having the MTHFR T allele in addition to the s allele of collagen will show a higher risk of fracture compared to a subject having the MTHFR C allele, which confers the lowest risk of fracture (see EXAMPLE 2).

These results could not be predicted, as previous studies have shown that these risk alleles predict fracture risk largely independent of bone mineral density. This fact is borne out by the results presented herein, which show that those individuals at highest risk of bone damage are not those having low bone mineral density. By screening for alleles of the MTHFR gene, susceptibility to bone damage may be assessed without the need for analysis of bone mineral density.

Thus, some embodiments provide methods for determining susceptibility to bone damage in a subject, comprising determining the presence of the T allele of the C677T polymorphism of the MTHFR gene and/or the s allele of the SpI polymorphism of the collagen Iα1 gene, wherein the presence of at least one copy of the T allele of the C677T polymorphism of the MTHFR gene and at least one copy of the s allele of the SpI polymorphism of the collagen Iα1 gene in the subject is indicative of an increased susceptibility to bone damage.

Typically, the method of the first aspect of the present invention comprises analysis of polymorphisms in collagen Iα1 and MTHFR to determine susceptibility to bone damage. The method may include analysis of DNA, RNA or protein, to determine which allele of a polymorphism is present. The method may include determining whether one or more particular alleles are present. The method may further comprise determining whether subjects are homozygous or heterozygous for alleles of collagen Iα1 and MTHFR.

Preferably, the method of the first aspect of the present invention further comprises determining whether the alleles that are present are associated with risk of bone damage. This may be performed by comparing the alleles present in a subject with those known to be associated with risk of bone damage. For example, a visual aid detailing alleles and the relative risk of bone damage associated therewith may be used to determine whether the genotype or phenotype of the subject is associated with a high or low risk of bone damage.

The methods of the present invention may be performed in vitro. Preferably, the method is performed on a tissue or fluid sample removed from the body of the subject. Thus, the present invention relates to a non-invasive diagnostic method, the results of which provide an indication of susceptibility to bone damage but do not lead to a diagnosis upon which an immediate medical decision regarding treatment has to be made.

The present invention may be performed on any living subject for which it is desirable to determine risk of bone damage. Preferably, the subject is a mammal. Most preferably, the subject is a human, preferably a female.

An alternative embodiment of the first aspect provides a method for determining susceptibility of a subject to bone damage, the method comprising determining in a subject having an s allele at the Sp1 polymorphism of collagen Iα1, the presence of an allele of an MTHFR polymorphism. Thus, in some embodiments the method comprises determining which allele(s) of an MTHFR polymorphism are present in a subject having an s allele at the Sp1 polymorphism of collagen Iα1, wherein the MTHFR polymorphism is associated with an increased or a decreased susceptibility to bone damage in a subject having an s allele at the Sp1 polymorphism of collagen Iα1. For example, the method may comprise determining which allele of the C677T polymorphism in MTHFR is present in a subject having an s allele at the Sp1 polymorphism of collagen Iα1, wherein the presence of the T allele of the C677T polymorphism in MTHFR is associated with an increased susceptibility to bone damage in a subject having an s allele at the Sp1 polymorphism of collagen Iα1. In a preferred embodiment, the method may comprise the step of determining which allele of the Sp1 polymorphism is present in the subject.

In a preferred embodiment of the first aspect of the present invention, the method comprises analyzing the genetic material of a subject to determine which allele(s) of the C677T polymorphism of MTHFR and which allele(s) of the Sp1 polymorphism of collagen Iα1 are present. The subject may be further classified as heterozygous or homozygous for each allele. Preferably, the method comprises the additional step of determining whether the alleles present are associated with risk of bone damage, wherein in a subject having the collagen Iα1 s allele, the presence of the MTHFR T allele is associated with increased risk of bone damage, and presence of the MTHFR C allele is associated with reduced risk of bone damage. Homozygosity for the T allele may further increase the susceptibility to bone damage in a subject having the collagen Iα1 s allele, while homozygosity for the C allele may further decrease susceptibility in a subject having the collagen Iα1 s allele. Thus, in some embodiments the method comprises determining which alleles of the C677T polymorphism in MTHFR are present in a subject having an s allele at the Sp1 polymorphism of collagen Iα1, wherein the presence of two T alleles of the C677T polymorphism in MTHFR is associated with a higher susceptibility to bone damage in a subject having an s allele at the Sp1 polymorphism of collagen Iα1 than the presence of at least one C allele of the C677T polymorphism in MTHFR.

Thus, homozygosity for the C allele may be considered to be protective against bone damage in a subject having an s allele at the Sp1 polymorphism of collagen Iα1. Thus, another embodiment of the present invention provides a method for determining decreased susceptibility to bone damage by screeening for the C allele of the C677T polymorphism of MTHFR in a subject having an s allele at the Sp1 polymorphism of collagen Iα1. Thus, in some embodiments the method comprises determining which alleles of the C677T polymorphism in MTHFR are present in a subject having an s allele at the Sp1 polymorphism of collagen Iα1, wherein the presence of two C alleles of the C677T polymorphism in MTHFR is associated with a lower susceptibility to bone damage in a subject having an s allele at the Sp1 polymorphism of collagen Iα1 than the presence of at least one T allele in a subject having an s allele at the Sp1 polymorphism of collagen Iα1.

In another preferred feature of the first aspect, the method may include analyzing the MTHFR protein of a subject to determine which allele of the Ala222Val polymorphism is present. Again, the method may further comprise the additional step of determining whether the allele present is associated with increased risk of bone damage, wherein presence of a valine residue at position 222 is indicative of increased risk of bone damage in a subject having an s allele at the Sp1 polymorphism of collagen Iα1, and the presence of an alanine residue at this position is indicative of reduced or normal risk of bone damage in a subject having an s allele at the S1 polymorphism of collagen Iα1.

Another preferred embodiment of the first aspect provides a method for determining susceptibility to bone damage, comprising determining the copy number of the alleles of the Sp1 polymorphism of the collagen Iα1 gene and the C677T polymorphism of MTHFR, where an increase in copy number of the T allele of the C677T polymorphism of MTHFR and increase in copy number of the s allele of the Sp1 polymorphism of the collagen Iα1 gene is associated with increased susceptibility to bone damage.

The present invention may also comprise screening for other polymorphisms in the MTHFR gene and/or the collagen Iα1 gene that may be useful in determining risk of fracture. In some embodiments, the method includes the step of determining that an allele of at least one polymorphism in a collagen Iα1 gene and/or an MTHFR gene is associated with an increased or a decreased susceptibility to bone damage. Exemplary methods for screening for polymorphisms in these genes are provided in EXAMPLE 2. The determination that an allele of a polymorphism in a collagen Iα1 gene and/or an MTHFR gene is associated with an increased or a decreased susceptibility to bone damage may be performed using standard statistical analyses in a population of subjects, as described for example in EXAMPLE 2.

The present invention may be performed using any suitable method known in the art. Preferably, a tissue or fluid sample is first removed from a subject. Examples of suitable samples include blood, mouth or cheek cells, and hair samples containing roots. Other suitable samples would be known to the person skilled in the art. The genetic material or protein is then extracted from the sample, using any suitable method. The genetic material may be DNA or RNA, although preferably DNA is used. For example, the genetic material or protein may be extracted using the techniques described in Sambrook et al. (Molecular Cloning—A Laboratory Manual, Cold Spring Harbor Laboratory Press). Determination of the genotype or phenotype of a subject may then be carried out using the extracted DNA or protein, employing any suitable technique, including, for example, Southern blot analysis followed by restriction enzyme digestion; PCR amplification followed by restriction enzyme digestion and, optionally, separation of digestion products by gel electrophoresis; sequencing of a relevant gene fragment by any suitable method; visualization of heteroduplex patterns, for example on PAA or agarose gels, where different patterns may indicate the presence of one or more specific alleles; separation of DNA fragments using denaturing gradient gels, wherein the degree of separation will depend upon the presence or absence of one or more polymorphic restriction sites; separation using SSCP analysis, the patterns of which will depend upon the presence or absence of one or more polymorphic restriction sites; use of allele specific oligonucleotides, hybridization patterns of which will be specific for various combinations of alleles; methods such as OLA, Taqman or dot-blot for the detection of known mutations; visualization of DNA sites using fluorescent labeled probes for alleles of interest; and RFLP analysis.

Where protein is to be analyzed, suitable methods may include the use of antibodies which are capable of distinguishing between different polymorphic forms of the protein; immunoassays; mobility shift assays, or other techniques capable of detecting differences in protein size; and assays for detecting changes in protein activity.

Where it is desirable to use particular restriction enzymes in performing the present invention, the skilled person will understand that enzymatic or chemical procedures having similar specificities may also be used. For example, restriction enzymes having similar specificity (isoschizomers) to those described herein may be used, or chemical degradation procedures with DNA or RNA cutting specificity.

Other techniques suitable for determining the genotype or phenotype of a subject may be used in the present invention.

Amplification is preferably carried out by polymerase chain reaction (PCR) techniques, to produce copies which, where the fragment is of the MTHFR, are at least about 20, preferably at least about 50, about 70, about 100, about 150, or about 200 bases in length. Where the fragment to be amplified is of the collagen Iα1 gene, PCR primers may be selected to amplify a fragment which is at least about 50 base pairs in length, preferably at least 200 base pairs in length.

Exemplary PCR primers are at least about 10 nucleotides in length, preferably at least about 15 nucleotides or at least about 20 nucleotides in length, and are complementary to any stretch of at least about 10 nucleotides of the sequence to be amplified. PCR techniques are well known in the art, and it is within the ambit of the skilled person to identify primers for amplification of the appropriate region of the above genes, namely the region from nucleotides 170 to 1100 of the MTHFR gene and the first intron of the collagen Iα1 gene. A preferred technique is single base extension, and for this method it is only necessary to amplify a fragment including the polymorphic site. Thus, amplification of the region immediately surrounding the C677T polymorphic site is required. Exemplary PCR techniques are described in EP-A-0200362 and EP-A-0201184.

In a preferred feature of the first aspect, there is provided a method for determining susceptibility to bone damage in a subject, said method comprising amplifying a fragment comprising a portion of the region from nucleotides 170 to 1100 of the MTHFR gene, and determining which allele(s) in the MTHFR is/are present. Primers suitable for amplification of said portion of the MTHFR gene would be readily available to a person skilled in the art. Examples of such primers include:

(SEQ ID NO: 4)
1. 5′ - TGAAGGAGAAGGTGTCTGCGGGA - 3′
and/or
(SEQ ID NO: 5)
2. 5′ - AGGACGGTGCGGTGAGAGTG - 3′.

To determine which allele of the Sp1 polymorphism of the collagen Iα1 gene is present, at least a portion of the first intron of the collagen Iα1 gene may be amplified, followed by determination of the presence of a MscI restriction site. Suitable primers include:

(SEQ ID NO: 6)
1. 5′-TAACTTCTGGACTATTTGCGGACTTTTTGG-3′
and/or
(SEQ ID NO: 7)
2. 5′-GTCCAGCCCTCATCCTGGCC-3′

Additional primer sequences are described in Grant et al. (1996) (Nature 14:203-205).

In a second aspect, the invention provides a method for determining risk of bone damage, comprising measuring the level of serum homocysteine in a subject, wherein the presence of an elevated level of serum homocysteine compared to the level of serum homocysteine in a reference population is indicative of an increased risk of bone damage. In some embodiments, the method comprises measuring the level of serum homocysteine in a subject, wherein a level of serum homocysteine that is equal or greater to the upper quartile level of serum homocysteine in the reference population is indicative of an increased susceptibility to bone damage. The reference population is typically a population of living organisms of the same species and sex as the living subject whose risk of bone damage is being determined. In some embodiments, the method comprises measuring the level of serum homocysteine in a subject, wherein a level of serum homocysteine that is greater than about 20 μmol/l is indicative of an increased susceptibility to bone damage.

A representative method for determining serum homocysteine levels is provided in EXAMPLE 1. Thus, total homocysteine may be determined as a fluorescence derivative, using high pressure liquid chromatography according to Araki & Sako (1987) (J. Chromatogr. 422:43-52), and modified by Ubbink et al. (1991) (J. Chromatogr. 565:441-446).

A representative method for stratifying homocysteine values in a reference population into quarters is provided in EXAMPLE 1. Susceptibility to bone damage may be determined using standard statistical analyses in a population of subjects, as described, for example, in EXAMPLE 1. Thus, Cox proportional-hazard models may be used to estimate non-vertebral fracture risks and logistic regression models may be used to estimate the risk of vertebral fractures. In some embodiments, a homocysteine level in the highest age-specific quartile attributes 14% to the risk of non-vertebral fracture in the population.

The third aspect of the present invention provides a method for preventing or reducing risk of bone damage in a subject. In some embodiments, the subject is diagnosed as being at risk of bone damage, preferably using the methods for the first aspect of the present invention. In this aspect, prevention or reduction in risk of bone damage includes any means of reducing the risk of bone damage in a subject.

In some embodiments, the method for preventing or reducing bone damage in a subject comprises the steps of: (1) determining that a subject has an increased susceptibility to bone damage by determining the presence in the subject of allele(s) of polymorphisms of an MTHFR gene and a collagen Iα1 gene that are associated with increased susceptibility to bone damage; and (2) prescribing or administering therapy to the subject that reduces the risk of susceptibility of the subject to bone damage. The polymorphism in the MTHFR gene that is associated with an increased risk of bone damage may be the C677T polymorphism. The polymorphism in the collagen Iα1 gene that is associated with an increased risk of bone damage may be the SpI polymorphism.

In some embodiments, the method for preventing or reducing bone damage comprises the steps of: (1) determining the presence of an elevated level of serum homocysteine in a subject, wherein the presence of an elevated level of serum homocysteine compared to the level of serum homocysteine in a reference population is indicative of an increased risk of bone damage; and (2) prescribing or administering therapy that reduces the risk of susceptibility of a subject to bone damage. The reference population is typically a population of living organisms of the same species and sex as the living subject whose risk of bone damage is being determined.

Therapy may be in the form of preventative or palliative care. A preferred method of treatment is prescribing or administering an agent that reduces the susceptibility of a subject to bone damage. For example, the treatment may comprise prescribing or administering folic acid, or folate, in order to reduce homocysteine levels in the plasma, and effectively reverse the effect of the MTHFR risk allele. Other suitable treatments which may be prescribed or administered alongside folic acid, or as an alternative thereto, include modifications to lifestyle, regular exercise and changes in diet to strengthen bones, and hormone therapy. Other suitable treatments, including pharmaceutical preparations to reduce bone loss, would be known to physicians and persons skilled in the art. Examples include anabolic steroids, bisphosphonates, vitamin D preparations, calcium supplements and Hormone Replacement Therapy. In a preferred embodiment of the third aspect there is provided the use of folic acid in the manufacture of a medicament for use in the prevention of bone damage.

Some embodiments provide methods for preventing or reducing bone damage, comprising the steps of: (1) determining that a subject has an increased susceptibility to bone damage by a method comprising the step of determining the presence in the subject of allele(s) of polymorphisms in an MTHFR gene and a collagen Iα1 gene that are associated with increased susceptibility to bone damage; and (2) prescribing or administering folic acid to the subject. The polymorphism in the MTHFR gene that is associated with an increased risk of bone damage may be the C677T polymorphism. The polymorphism in the collagen Iα1 gene that is associated with an increased risk of bone damage may be the SpI polymorphism.

In some embodiments, the method for preventing or reducing bone damage comprises the steps of: (1) determining that a subject has an increased susceptibility to bone damage by a method comprising the step of determining the presence in a subject of an elevated level of serum homocysteine, wherein the presence of an elevated level of serum homocysteine compared to the level of serum homocysteine in a reference population is indicative of an increased risk of bone damage; and (2) prescribing or administering folic acid to the subject. The reference population is typically a population of living organisms of the same species and sex as the living subject whose risk of bone damage is being determined.

Administration of the medicament is accomplished by any effective route, e.g., orally or parenterally. Methods for parenteral delivery include topical, intra-arterial, subcutaneous, intramedullary, intravenous, or intranasal administration. Oral administration followed by subcutaneous injection would be the preferred routes of uptake; also long acting immobilizations would be used. In addition to the active ingredients, these medicaments may contain suitable pharmaceutically acceptable carriers comprising excipients and other compounds that facilitate processing of the active compounds into preparations which can be used pharmaceutically. Further details on techniques for formulation and administration may be found in the latest edition of “Remington's Pharmaceutical Sciences” (Mack Publishing Co, Easton Pa.).

Medicaments for oral administration can be formulated using pharmaceutically acceptable carriers well known in the art, in dosages suitable for oral administration. Such carriers enable the pharmaceutical compositions to be formulated as tablets, pills, dragees, capsules, liquids, gels, syrups, slurries, suspensions, etc., suitable for ingestion by the patient.

Medicaments for oral use can be obtained through combination of active compounds with solid excipient, optionally grinding a resulting mixture, and processing the mixture of granules, after adding suitable additional compounds, if desired, to obtain tablets or dragee cores. Suitable excipients are carbohydrate or protein fillers. These include, but are not limited to sugars, including lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins, such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Medicaments, which can be used orally, include push-fit capsules made of gelatin, as well as soft, sealed capsules made of gelatin and a coating such as glycerol or sorbitol. Push-fit capsules can contain active ingredients mixed with filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Medicaments for parenteral administration include aqueous solutions of active compounds. For injection, the medicaments of the invention may be formulated in aqueous solutions, preferably in physiologically compatible buffers such as Hank's solution, Ringer's solution, or physiologically buffered saline. Aqueous injection suspensions may contain substances, which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents, which increase the solubility of the compounds to allow for the preparation of highly concentrated solutions.

For topical or nasal administration, penetrants appropriate to the particular barrier to be permeated are used in the formulation. Such penetrants are generally known in the art.

The medicaments of the present invention may be manufactured in a manner similar to that known in the art (e.g., by means of conventional mixing, dissolving, granulating, dragee-making, levigating, emulsifying, encapsulating, entrapping or lyophilizing processes). The medicaments may also be modified to provide appropriate release characteristics, e.g., sustained release or targeted release, by convention means, e.g., coating.

The medicaments may be provided as a salt and can be formed with many acids, including but not limited to hydrochloric, sulfuric, acetic, lactic, tartaric, malic, succinct, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5, that is combined with buffer prior to use.

After such medicaments formulated in an acceptable carrier have been prepared, they can be placed in an appropriate container and labeled for treatment of an indicated condition.

Medicaments suitable for prevention or reduction of bone damage include compositions wherein the active ingredients are contained in an effective amount to achieve the intended purpose. The amount actually administered will be dependent upon the individual to which treatment is to be applied, and will preferably be an optimized amount such that the desired effect is achieved without significant side-effects. The determination of a therapeutically effective dose is well within the capability of those skilled in the art. Of course, the skilled person will realize that divided and partial doses are also within the scope of the invention.

For any compound, the therapeutically effective dose can be estimated initially either in cell culture assays or in any appropriate animal model (e.g., primate, rats and guinea pigs for hypertension and other small laboratory animals). These assays should take into account receptor activity as well as downstream processing activity. The animal model is also used to achieve a desirable concentration range and route of administration. Such information can then be used to determine useful doses and routes for administration in humans.

A therapeutically effective amount refers to that amount of agent, which ameliorates the symptoms or condition. Therapeutic efficacy and toxicity of such compounds can be determined by standard pharmaceutical procedures, in cell cultures or experimental animals (e.g., ED₅₀, the dose therapeutically effective in 50% of the population; and LD₅₀, the dose lethal to 50% of the population). The dose ratio between therapeutic and toxic effects is the therapeutic index, and it can be expressed as the ration ED₅₀/LD₅₀. Medicaments, which exhibit large therapeutic indices, are preferred. The data obtained from cell culture assays and animal studies is used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Long acting medicaments might be administered every 3 to 4 days, every week, or once every two weeks depending on half-life and clearance rate of the particular formulation. Guidance as to particular dosages and methods for delivery is provided in the literature (see U.S. Pat. Nos. 4,657,760; 5,206,344 and 5,225,212, herein incorporated by reference).

In a fourth aspect of the present invention, there is provided a method for predicting the response of a subject to treatment to reduce the risk of bone damage. In some embodiments, the method comprises determining which allele(s) of a polymorphism of MTHFR and/or collagen Iα1 is/are present in a subject. In some embodiments, the method comprises measuring the level of serum homocysteine in a subject. This may be done according to the methods provided in the first aspect of the invention. Preferably, the method includes determining whether the subject is susceptible to bone damage.

The effect of a therapeutic or preventative agent may depend on the underlying cause of the bone damage, and in some cases it may be preferable to avoid the use of certain treatments for example, the presence or absence of particular alleles of a gene will provide a useful indication as to which is the most appropriate preventative measure. For example, a subject having a collagen Iα1 risk allele but no MTHFR risk allele is unlikely to benefit from folic acid administration. In contrast, a subject having an elevated level of serum homocysteine compared to a reference population is likely to benefit from folic acid, or folate, administration in order to reduce homocysteine levels in the plasma. This aspect of the present invention may also be useful for identifying agents which may be used in the treatment of bone damage.

Thus, in some embodiments, the present invention provides methods for predicting the response of a subject to treatment with folic acid that comprise the steps of: (1) determining which allele(s) of polymorphisms in an MTHFR gene are present in a subject; and (2) predicting the response of the subject to treatment with folic acid, wherein the absence of an allele of a polymorphism in an MTHFR gene that is associated with an increased risk of bone damage is indicative that treatment of the subject with folic acid is unlikely to reduce the risk of bone damage. The polymorphism in the MTHFR gene that is associated with an increased risk of bone damage may be the C677T polymorphism.

In some embodiments, the method for predicting the response of a subject to treatment with folic acid comprises the steps of: (1) determining that a subject has an increased susceptibility to bone damage by using a method comprising the step of determining the presence in the subject of one or more allele(s) of polymorphisms in an MTHFR gene and a collagen Iα1 gene that are associated with increased susceptibility to bone damage; and (2) predicting the response of the subject to treatment with folic acid, wherein the absence of an allele of a polymorphism in MTHFR that is associated with an increased risk of bone damage is indicative that treatment of the subject with folic acid is unlikely to reduce the risk of bone damage. The polymorphism in the MTHFR gene that is associated with an increased risk of bone damage may be the C677T polymorphism. The polymorphism in the collagen Iα1 gene that is associated with an increased risk of bone damage may be the SpI polymorphism.

In some embodiments, the method for predicting the response of a subject to treatment with folic acid comprises the steps of: (1) determining the presence of an elevated level of serum homocysteine in a subject, wherein the presence of the elevated level of serum homocysteine compared to the level of serum homocysteine in a reference population is indicative of an increased risk of bone damage; and (2) predicting the response of the subject to treatment with folic acid, wherein the presence of an elevated level of serum homocysteine in the subject is indicative that treatment of the subject with folic acid is likely to reduce the risk of bone damage.

In a fifth aspect of the present invention, there is provided a kit for use in determining which allele of a polymorphism of the MTHFR gene and/or collagen Iα1 gene are present, comprising (i) one or more nucleic acid primer molecules for amplification of a portion of the MTHFR and/or collagen Iα1 genes, and (ii) means for determining which allele(s) are present in those genes. Preferably, the polymorphisms of the MTHFR and/or collagen Iα1 genes are the C677T polymorphism and the SpI polymorphism, respectively. A kit for protein analysis may comprise antibodies capable of distinguishing between different polymorphic forms of a protein, and reagents necessary to carry out mobility shift or immunoassays.

In some embodiments, the kit provides a method for determining susceptibility to bone damage by determining the presence of alleles of MTHFR and/or collagen Iα1 that are associated with an increased or a decreased susceptibility to bone damage. In further embodiments, the kit may be used in a method for predicting the response of a subject to treatment by determining the presence of alleles of MTHFR and/or collagen Iα1 that are associated with an increased or a decreased susceptibility to bone damage.

Preferably, the kit also comprises means for indicating correlation between the allele(s) and risk of bone damage, or for predicting the response of a subject to treatment. Thus, the kit may contain written indicia providing information to the user for interpreting the results of the analysis. For example, the written indicia may explain that the presence of the MscI restriction site at the SpI polymorphism of the collagen Iα1 gene in a subject indicates the presence of the T allele at this polymorphic site.

Preferably, the primer molecules are suitable for amplification of at least a portion of the MTHFR gene, and/or a portion of the first intron of the collagen Iα1 gene. Primers suitable for amplification of a portion of the MTHFR gene would be readily available to a person skilled in the art. Examples of suitable primers are described above. For example, suitable primers may amplify a fragment comprising a portion of the region from nucleotides 170 to 1100 of the MTHFR gene, and determining which allele(s) of the C677T polymorphism in MTHFR is/are present. Examples of such primers include:

(SEQ ID NO: 4)
1. 5′ - TGAAGGAGAAGGTGTCTGCGGGA - 3′
and/or
(SEQ ID NO: 5)
2. 5′ - AGGACGGTGCGGTGAGAGTG - 3′.

Primers suitable for amplification of a portion of the collagen Iα1 gene would also be readily available to a person skilled in the art. For example, suitable primers may amplify at least a portion of the first intron of the collagen Iα1 gene to determine which allele of the SpI polymorphism of the collagen Iα1 gene is present. Suitable primers include:

(SEQ ID NO: 6)
1. 5′-TAACTTCTGGACTATTTGCGGACTTTTTGG-3′
and/or
(SEQ ID NO: 7)
2. 5′-GTCCAGCCCTCATCCTGGCC-3′

Additional primer sequences are described in Grant et al. (1996) (Nature 14:203-205).

Means for determining which allele(s) is/are present in the MTHFR gene, and/or collagen Iα1 gene may include any reagents or molecules necessary for use in any of the methods described above. For example, where PCR followed by DNA digestion is used, said means preferably include PCR reagents and one or more of the Hinf1 and/or MscI restriction enzymes. Where the method employs Southern Blotting, heteroduplex visualization, or fluorescent labeling techniques for example, probes which bind to the appropriate regions of the MTHFR gene, and/or collagen Iα1 gene may be included. Where necessary, such probes may be labeled to allow detection, for example by nick-translation, radio- or fluorescent-labeling, or random primer extension whereby the non-labelled nucleotides serve as a template for the synthesis of labeled molecules. Other methods for labeling probes are well known in the art.

The means for correlating the allele present with risk of bone damage, or for predicting response to treatment, may be in the form of a chart or visual aid. The chart or visual aid may indicate that presence of the T allele of the MTHFR polymorphism and the s allele of the collagen Iα1 gene is associated with increased risk of bone damage. The chart or visual aid may indicate that presence of the C allele of the MTHFR polymorphism predicts that the subject is unlikely to respond to treatment with folic acid.

In a preferred feature of the fifth aspect, the kit may also comprise control DNA or protein samples, for comparison with DNA sequences of a subject. The control samples may comprise the sequence of one or more alleles of the MTHFR and/or collagen Iα1 genes.

The following examples are provided for the purpose of illustrating, not limiting, the present invention.

### EXAMPLE 1

This Example describes a study examining the relationship between serum homocysteine levels in postmenopausal women, incidence of fracture, and bone marrow density (BMD).

### 1. Methods

A. Study Subjects

The Rotterdam Study is a population-based cohort study of 7983 subjects aged 55 years or more, residing in the Ommoord district of the city of Rotterdam in the Netherlands. The study was designed to document the occurrence of disease in the elderly in relation to several potential determinants (Hofman et al. (1991) Eur. J. Epidemiol. 7:403-422). A total of 10,275 persons, of whom 9161 (89%) were living independently, were invited to participate in the study in 1991. In those subjects living independently, the overall response rate was 77% for home interview and 71% for examination in a research center, including measurement of anthropometric characteristics, BMD, and blood sampling. The Rotterdam Study was approved by the Medical Ethics Committee of the Erasmus University Medical School and written informed consent was obtained from each subject. The analysis of the association between homocysteine, MTHFR genotype, COL1A1 genotype, BMD, and fractures was performed in a sample of women participating in the study.

The analysis of the association between homocysteine levels, fracture risk and BMD was done in a random sample of 459 women. In this subset, follow-up data of non-vertebral fractures were available for 428 women, and vertebral fracture date for 264 women.

B. Measurements

Anthropometric measures and BMD were measured as described previously (Burger et al. (1994) Bone Miner. 25:1-13). Height and weight were examined at the initial examination in standing position without shoes and indoor clothing. Body mass index (BMI) was computed as weight in kilograms divided by height in meters squared (kg/m²). Bone mineral density (BMD) was determined by dual energy X-ray absorptiometry (DEXA) (Lumar DPX-L densitometer, Luwar Corp., Madison, Wis., USA) at the femoral neck and lumbar spine (vertebrae L2-L4) as described previously (Burger et al. (1994) Bone Miner. 25:1-13). Dietary intakes of calcium (mg/day) and folate (μg/day) during the preceding year were assessed by food frequency questionnaire and adjusted for energy intake. Current cigarette smoking status, use of a walking aid, and falling in the preceding year was assessed by questionnaire. Serum creatinine was measured with the Jaffe method on a Hitachi 747 automated analyzer.

For 956 women (62%), lateral radiographs of the spine from the fourth thoracic to the fifth lumbar vertebrae were obtained both at baseline examination (between 1990 and 1993) and at a follow-up visit (between 1997 and 1999). All follow-up radiographs were scored for the presence of vertebral fractures by the McCloskey/Kanis method (McCloskey et al. (1993) Osteoporosis Int. 3:138-147). If a vertebral fracture was present, the baseline radiograph was scored as well, to ascertain whether a fracture was incident or prevalent.

The occurrence of incident nonvertebral fractures, including hip, wrist and other fractures, was recorded, confirmed, and classified by a physician over a mean follow-up period of 7 years. Fractures of the skull and head, and hand, and pathological fractures were not included. Follow-up started either at Jan. 1^{st}, 1991 or, when later, at the time of inclusion into the study. For this study follow-up ended at Dec. 31, 1999, or, when earlier, at the death.

C. Homocysteine Determination

Nonfasting serum samples were obtained at the baseline examination. The samples were put on ice immediately and were processed within 60 minutes, which has been shown to be sufficient to prevent increases in total homocysteine concentration due to ex vivo generation (Ubbink et al. (1992) Clin. Chim. Acta 207:119-128). Serum was kept frozen at −20° C. until determination of total homocysteine. Total homocysteine was determined as a fluorescence derivative, using high pressure liquid chromatography according to Araki & Sako (1987) (J. Chromatogr. 422:43-52), and modified by Ubbink et al. (1991) (J. Chromatogr. 565:441-446).

D. Data Analysis

The distribution of serum homocysteine levels was skewed; therefore natural-log-transformed values were used when the homocysteine levels were analyzed as a continuous variable.

Homocysteine values were also stratified into quartiles for each five-year category. The cut-off point for the third quartile was 14.6 μmol/liter for subjects aged 55-60, 15.4 μmol/liter for ages 60-65, 15.9 μmol/liter for ages 65-70, 18.5 μmol/liter for ages 70-75, 17.7 μmol/liter for ages 75-80, 17.7 μmol/liter for ages 80-85 and 24.1 μmol/liter for subjects aged over 85 years. Differences in baseline characteristics were compared by analysis of covariance (ANCOVA), with age as covariate to adjust for possible confounding effects.

Cox proportional-hazard models were used to estimate non-vertebral fracture risks. To estimate the risk of vertebral fractures logistic regression models were used. All estimated fracture risks were adjusted for age. In additional analyses, adjustments were also made for BMI, dietary intake of calcium and folate, smoking status, creatinine serum levels, use of a walking aid and falling.

### 2. Results: Homocysteine Levels, BMD, and Fracture Risk

Baseline characteristics of the 459 women in this study are presented in Table 1. Women in the upper age-specific quartile of homocysteine serum level smoked more, had higher serum creatinine levels, and their mean dietary calcium and folic acid intake was lower. The women in the upper quartile did not differ significantly with respect to BMD at the femoral neck and at the lumbar spine.

TABLE 1
Baseline Characteristics of 459 Postmenopausal
Women From the Homocysteine Serum Level Study*

| | | Age specific homocysteine quartiles | | |
|---|---|---|---|---|
| variable | All women | Quartile 1, 2 and 3 | Quartile 4 | p-value† |
| Number of women | 459 | 345 | 114 | |
| Homocysteine level (μmol/liter) | 15.2 ± 5.8 | 13.2 ± 2.7 | 21.4 ± 8.0 | <0.001 |
| Age (yr) | 69.6 ± 8.9 | 69.6 ± 8.7 | 69.7 ± 9.3 | 0.89 |
| BMI (kg/m²) | 27.1 ± 4.0 | 27.0 ± 4.2 | 27.5 ± 3.5 | 0.25 |
| Smoking status (%) | 18 | 16 | 25 | 0.04 |
| Ca dietary intake (mg/day) | 1049 ± 326 | 1096 ± 302 | 954 ± 308 | <0.001 |
| Folate dietary intake(μg/day) | 203 ± 62 | 208 ± 64 | 187 ± 53 | 0.004 |
| Creatinine serum level (μmol/l) | 76.7 ± 14.2 | 75 ± 13 | 84 ± 31 | <0.001 |
| Fall in preceding year | 24 | 24 | 23 | 0.86 |
| Use of a walking aid (%) | 14 | 13 | 25 | 0.08 |
| BMD femoral neck (g/cm²) | 0.83 ± 0.13 | 0.82 ± 0.13 | 0.84 ± 0.10 | 0.45 |
| BMD lumbar spine (g/cm²) | 1.05 ± 0.19 | 1.05 ± 0.19 | 1.05 ± 0.17 | 0.72 |

*Values are proportions or means ± standard deviations.
†P-values are calculated with analysis of variance (ANOVA) testing differences between quartile 4 and all other women.

Table 2 shows the results of the association analysis of homocysteine serum level with fracture risk in 428 women for whom data are available. After adjustment for potential confounders, the relative risk for non-vertebral fracture was 1.4, and the odds ratio for vertebral fracture risk was 2.0 for each increase of one SD in log-transformed homocysteine serum level.

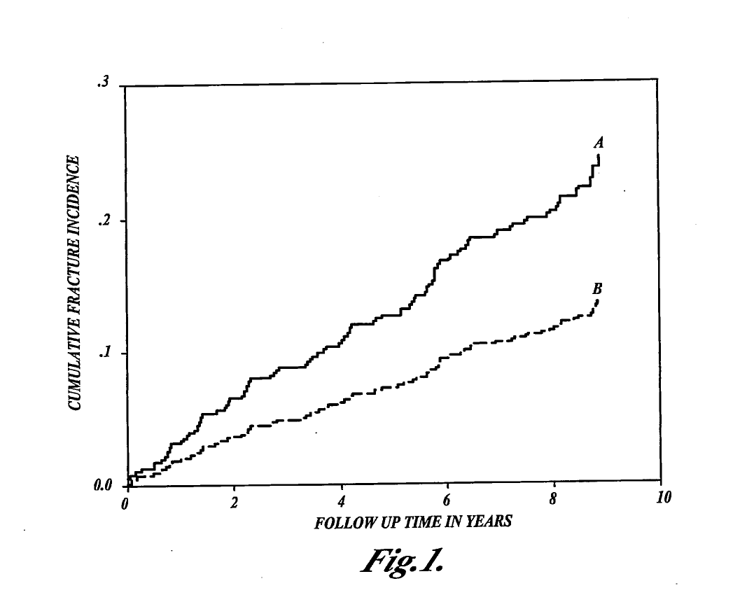

FIG. 1 shows the cumulative incidence of non-vertebral fractures according to the upper age-specific quartile of homocysteine levels. Women in the upper quartile had significantly more non-vertebral fractures compared to all other women, corresponding to a relative risk of 1.9 (95% CI 1.1-3.3) after adjustment for age. After additional adjustment for BMI, dietary intake of calcium and folate, smoking status, creatinine serum level, use of a walking aid and falling, the relative risk remained unaltered and was 1.9 (95% CI 1.0-3.6) for women in the upper quartile as compared to the other women. In this population the risk of fracture attributable to a homocysteine level in the highest age-specific quartile was 14 percent.

### 3. Conclusions

The data show, for the first time, an association between homocysteine serum levels and fracture risk in postmenopausal women. A serum homocysteine level in the highest quartile doubled the risk for non-vertebral fractures, and this increased risk appeared to be independent of age or other confounding factors. The magnitude of this effect is similar to what was found previously for the increase in risk of cardiovascular disease and dementia (Boushey et al. (1995) J.A.M.A. 274:1049-57; Welch & Loscalzo (1998) N. Engl. J. Med. 338:1042-50; Clarke et al. (1998) Arch. Neurol. 55:1449-55; Seshadri et al. (2002) N. Engl. J. Med. 346:476-83). A homocysteine level in the highest age-specific quartile attributed 14% to the risk of non-vertebral fracture in the population.

TABLE 2
Multivariate Regression Models Examining the Relation
Between Baseline Serum Homocysteine Levels and
Fracture risk in 428 Postmenopausal Women*

| | | Variables adjusted for | |
|---|---|---|---|
| | | Age | Multivariate† |
| Non-vertebral | n cases/n subjects | 57/428 | 48/372 |
| | RR [95% CI] | 1.2 [0.9-1.6] | 1.4 [1.0-1.9] |
| | p-value‡ | 0.2 | 0.08 |
| vertebral | n cases/n subjects | 21/264 | 19/250 |
| | OR [95% CI] | 1.8 [1.0-3.0] | 2.0 [1.1-3.7] |
| | p-value§ | 0.04 | 0.03 |
| Any fracture | n cases/n subjects | 47/264 | 43/250 |
| | OR [95% CI] | 1.9 [1.2-2.8] | 2.4 [1.4-4.0] |
| | p-value§ | 0.005 | 0.001 |

*The serum homocysteine level was analyzed as a continuous variable. The relative risks (RRs) or odds ratios (ORs) were estimated per increment of 1 SD (=0.3) in the log-transformed value of homocysteine serum concentration; n = number.
†The multivariate analysis included adjustments for age, BMI, smoking status, dietary calcium intake, dietary folate intake, creatinine serum level and use of a walking aid.
‡P-values are calculated with Cox regression models
§P-values are calculated with logistic regression models

### EXAMPLE 2

This Example describes a study examining the interaction between polymorphisms in the COLIα1 gene and the MTHFR gene in relation to BMD and fracture risk.000

### 1. Methods

A. Study Subjects

For the analysis of the association between the MTHFR gene, the COLIα1 gene and fracture risk and BMD, a subgroup of women was studied. Baseline measurements of BMD were available for 5931 independently living subjects from the study, but 1453 of these were excluded based on use of a walking aid, diabetes mellitus, use of diuretic, estrogens, thyroid hormone, or cytostatic drug therapy. From remaining subjects, a random sample of 1533 women, aged 55-80 years was studied. Follow-up data for non-vertebral fractures and vertebral fractures was available for 1374 of these and 955 of these, respectively.

B. Measurements

Measurements were made as described in EXAMPLE 1, above.

C. Determination of COLIα1 and MTHFR Genotypes Genomic DNA was extracted from peripheral venous blood samples according to standard procedures and the polymorphism in the COLIα1 gene was detected by polymerase chain reaction (PCR) with a mismatched primer that introduces a diallelic restriction site, as described previously (Grant et al. (1996) Nature Genet. 14:203-5). The test discriminates two alleles, G and T, corresponding to the presence of guanine and thymidine respectively, at the first base of the Sp1 binding site in the first intron of the COLIα1 gene.

The C677T polymorphism in the MTHFR gene was detected by PCR-amplification of the DNA-fragment containing the polymorphism, after which the fragment was digested with HinfI, as described previously (Frosst et al. (1995) Nature Genet. 10:111-3). The test discriminates two alleles, C and T, corresponding to an alanine or valine respectively at codon 222 in the protein.

D. Data Analysis

Subjects were grouped according to genotype. To study the MTHFR and COLIα1 variants separately, subjects were grouped into reference, heterozygotes and homozygotes for the risk allele. To study interaction of both genetic variants, subjects were analyzed in four groups according to presence or absence of risk alleles. For reasons of power, heterozygous and homozygous subjects for the risk allele were combined into carriers of at least one copy of the risk allele.

Chi-square analysis was used to test for deviation from Hardy-Weinberg equilibrium. For exploring an association between BMD and different genotypes, we used multivariate linear regression models were used.

Fracture risks were estimated, designating subjects without a risk allele as the reference group. To estimate the risk of vertebral fractures, logistic regression analysis was used. To estimate non-vertebral fractures, Cox proportional hazard models was used.

### 2. Results: MTHFR and COLIα1 Polymorphisms and Osteoporosis

Baseline characteristics of the women in this genetic study are shown in Table 3. The women of the genetic study were on average 3.8 years younger compared to the women that were part of the homocysteine serum study.

Table 4 shows the genotype frequencies of the MTHFR Ala222Val polymorphism with corresponding distribution of fractures. The distribution of genotypes were similar to those reported for Caucasians in previous studies (Botto & Yang (2000) Am. J. Epidemiol. 151:862-877; Beavan et al. (1998) N. Engl. J. Med. 339:351-2; Langdahl et al. (1998) Miner. Res. 13:1384-9; Keen et al. (1999) Arthritis Rheum. 42:285-90), and did not deviate from Hardy-Weinberg Equilibrium (p=0.96). No significant differences between the three different MTHFR-genotype groups were found with respect to baseline age, height, weight, dietary calcium and folate intake, and smoking status. No significant association of the MTHFR variant was seen with either vertebral or non-vertebral fracture risk.

TABLE 3
Baseline Characteristics of 1532 Postmenopausal
Women from the Genetic Study*

| variable | |
|---|---|
| Age (yr) | 66.1 ± 6.8 |
| BMI (kg/m²) | 26.2 ± 3.8 |
| Smoking status (%) | 21 |
| Ca dietary intake (mg/day) | 1103 ± 325 |
| Folate dietary intake (μg/day) | 206 ± 68 |
| Creatinine serum level (μmol/l) | 76.2 ± 14.1 |
| BMD femoral neck (g/cm²) | 0.81 ± 0.12 |
| BMD lumbar spine (g/cm²) | 1.02 ± 0.16 |

*Values are proportions or means ± standard deviation
The mean age mean is a crude value, all other values are adjusted for age

Table 4 also shows BMD values according to the three MTHFR genotype groups. A significant allele-dose effect on BMD at femoral neck, and lumbar spine was observed. At both sites, BMD was 0.2 SD lower in the Val-Val genotype group compared to the Ala-Ala group, after adjustment for age and BMI.

For the COLIα1 Sp1 polymorphism, results were similar to previous observations in this population (Uitterlinden et al. (1998) N. Engl. J. Med. 338:1016-21). The COLIα1 Sp1 T-allele showed a significant association with non-vertebral fractures but no association was found with vertebral fractures. When designating the GG homozygotes as the reference group, the relative risk for women heterozygous for the Sp1 T-allele was 1.4 (95% CI 1.0-1.9), while for women homozygous for the Sp1 T-allele the relative risk was 2.0 (95% CI 1.0-3.9). An allele-dose effect was observed for the association of the Sp1 T-allele being with lower BMD at both the femoral neck and the lumbar spine.

TABLE 4
Number of Women With Fracture with Corresponding Risk
Estimates and BMD Measures According to MTHFR Genotype

| | | MTHFR genotype | | | |
|---|---|---|---|---|---|
| | | Ala-Ala | Ala-Val | Val-Val | p-value¶ |
| Non-vertebral fractures | n fractures/total n (%) | 66/601 (11) | 86/616 (14) | 22/158 (14) | 0.26 |
| | Relative Risks [95% CI]* | | | | |
| | Crude | 1.0 | 1.3 [0.9-1.8] | 1.3 [0.8-2.1] | |
| | Adjusted† | 1.0 | 1.3 [1.0-1.8] | 1.3 [0.8-2.1] | |
| Vertebral fractures | n fractures/total n (%) | 51/415 (12) | 54/442 (12) | 12/98 (12) | 1.00 |
| | OddsRatios [95% CI]* | | | | |
| | Crude | 1.0 | 1.0 [0.7-1.5] | 1.0 [0.5-1.9] | |
| | Adjusted† | 1.0 | 0.9 [0.6-1.4] | 1.0 [0.5-1.9] | |
| BMD femoral neck‡ | Mean values | | | | |
| | Crude | 0.82 ± 0.13 | 0.81 ± 0.12 | 0.80 ± 0.12 | 0.01 |
| | Adjusted§ | 0.82 ± 0.10 | 0.81 ± 0.10 | 0.80 ± 0.12 | 0.007 |
| BMD lumbar spine‡ | Mean values† | | | | |
| | Crude | 1.03 ± 0.17 | 1.01 ± 0.17 | 1.00 ± 0.17 | 0.008 |
| | Adjusted§ | 1.03 ± 0.16 | 1.02 ± 0.16 | 1.00 ± 0.17 | 0.006 |

*Relative Risks are estimated with Cox regression models; Odds Ratios are estimated with logistic regression models
†Relative risks and Odds ratios are adjusted for age and BMI.
‡Values are means ± standard deviation.
§BMD values are adjusted for age and BMI.
¶P-values for the distribution of fractures were calculated by chi-square analysis, p-values for association of BMD with MTHFR genotype were calculated with linear regression models.

Femoral neck BMD was 0.2 SD lower and lumbar spine BMD 0.4 SD lower in women with the TT genotype compared to the GG group.

To study interaction of both genetic variants, subjects were divided into four groups: a reference group without risk alleles for MTHFR and COLIα1, a group with presence of MTHFR risk allele(s), but no risk allele for COLIα1, a group with presence of risk allele(s) for COLIα1 but not for MTHFR, and a group with presence of risk allele(s) for both MTHFR and COLIα1. No significant differences in baseline anthropometric and dietary measurements for these four genotype groups were observed.

Table 5 shows the distribution of incident non-vertebral fractures in women grouped according to their combined MTHFR and COLIα1 genotype. An overrepresentation of non-vertebral fractures was observed in women carrying at least one risk allele for MTHFR and COLIα1. Women carrying both risk alleles have a 1.9 times increased risk for non-vertebral fractures, while subjects having only one of the risk alleles do not have a significantly increased fracture risk. After adjustment of the risk estimate for age, BMI and femoral neck BMD, the relative risk remained 1.7. No significant difference was seen between the four genotype groups with respect to the presence of vertebral fractures.

The fracture risk attributable to the combined presence of risk alleles for MTHFR and COLIα1 was 10% in the total study population.

The cumulative fracture incidence during the follow-up period according to combined MTHFR/COLIα1 genotype for all women in the genetic study population was calculated and found that the combined presence of risk alleles for MTHFR and COLIα1 resulted in the highest fracture incidence, as shown in FIG. 7. This effect was mainly present in older women. FIG. 8 shows the cumulative fracture incidence in women older than the median of 66 years. Cox regression showed women older than 66 years

TABLE 5
Number of Women With Non-Vertebral Fractures and Relative
Risks According to Combined MTHFR and COLIA1 Genotype*

| genotype | | n fractures/ | | | |
|---|---|---|---|---|---|
| MTHFR | COL1AI | total n (%) | crude | Multivariate 1† | Multivariate 2‡ |
| − | − | 41/412 (10.0) | 1.0 | 1.0 | 1.0 |
| + | − | 63/522 (12.1) | 1.2 [0.8-1.8] | 1.2 [0.8-1.8] | 1.2 [0.8-1.7] |
| − | + | 24/188 (12.8) | 1.3 [0.8-2.2] | 1.3 [0.8-2.2] | 1.2 [0.8-2.0] |
| + | + | 45/253 (17.8) | 1.9 [1.2-2.9] | 1.9 [1.2-2.9] | 1.7 [1.1-2.6] |

*Relative risks were calculated with Cox regression models.
†Multivariate 1: adjusted for age and BMI
‡Multivariate 2: adjusted for age, BMI and femoral neck BMD

and carrying both risk alleles to have a 2.5 (95% CI 1.5-4.2) times higher risk than the reference group. When this risk calculation was adjusted for age, BMI and femoral neck BMD, the fracture risk decreased somewhat and was 2.1 (95% CI 1.2-3.5), as compared to the reference group.

The relation between the combined MTHFR /COLIα1 genotype and BMD was investigated. Among the four combined MTHFR/COLIα1 genotype groups, BMD at both the femoral neck and the lumbar spine differed significantly, as shown in Table 6. At both sites, a lower BMD was seen when risk alleles at both gene loci were present, compared to the presence of a risk allele of either MTHFR or COLIα1 alone or when compared to the reference group carrying no risk alleles for both genes. The differences in BMD were more pronounced in older women.

### 3. Conclusions

Women carrying genetic risk variants for crucial genes in each of the pathways (i.e., MTHFR and COLIα1, respectively), have an almost two-fold increased fracture risk. This increase in fracture risk is largely independent of BMD differences. The presence of risk alleles at both the MTHFR and COLIα1 loci attributed 10% to the fracture risk. These data suggest an interaction of the homocysteine and collagen metabolic pathways. For comparison, a recent report showed that for the Rotterdam Study, population attributable risks of hypercholesterolemia and hypertension were 18% and 14%, respectively (De Laet et al. (1997) Br. Med. J. 315:221-5). Thus, the risk factors for fracture identified in the present study are similar in magnitude of effect to established risk factors for cardiovascular disease.

There was no association of the MTHFR 222-Val variant by itself with fracture risk, although this allele is associated with decreased BMD.

TABLE 6
BMD Measures in 1532 Postmenopausal Women According to Combined
MTHFR and COLIA1 Genotype*

| genotype† | | n | FN-BMD | | LS-BMD | |
|---|---|---|---|---|---|---|
| MTHFR | COL1AI | women | crude | Adjusted‡ | crude | Adjusted‡ |
| − | − | 461 | 0.83 ± 0.13 | 0.82 ± 0.11 | 1.04 ± 0.17 | 1.03 ± 0.17 |
| + | − | 576 | 0.81 ± 0.13 | 0.81 ± 0.12 | 1.02 ± 0.17 | 1.02 ± 0.17 |
| − | + | 214 | 0.81 ± 0.12 | 0.81 ± 0.12 | 1.03 ± 0.18 | 1.03 ± 0.16 |
| + | + | 281 | 0.79 ± 0.12 | 0.80 ± 0.12 | 0.99 ± 0.17 | 1.00 ± 0.17 |
| p-value§ | | | 0.002 | 0.009 | 0.005 | 0.01 |

*Values are means ± standard deviation. BMD, bone mineral density; FN, femoral neck; LS, lumbar spine.
†Women are grouped according to carrier status for the risk alleles for MTHFR and COLIA1. MTHFR − = Ala-Ala, MTHFR + = Ala-Val or Val-Val; COLIA1 − = GG, COLIA1 + = GT or TT.
‡BMD values are adjusted for age and BMI.
§P-values are estimated by ANOVA in case of crude values, and by ANCOVA in case of adjusted values.

### EXAMPLE 3

This Example describes a study examining the relationship between a polymorphism in the MTHFR gene, homocysteine levels, fracture risk, and BMD.

### 1. Methods

For a description of the methodology used, please see EXAMPLES 1 and 2, above.

### 2. Results: MTHFR Polymorphism and Homocysteine

In an analysis of the homocysteine levels according to MTHFR genotype, no differences were observed between homocysteine levels in the different genotype groups. One important factor in the association between the MTHFR-variant and homocysteine levels is the plasma folate status (Ma et al. (1996) Circulation 94:2410-6; Jacques et al. (1996) Circulation 93:7-9; Harmon et al. (1996) Quarterly J Med. 89:571-7). This is in part determined by dietary intake of folate, for which data were available. When mean homocysteine values were adjusted for dietary folate intake, 222 Val carriers were found to have a 0.7 μM/l higher homocysteine serum level compared to non-carriers, but this did not reach significance (p=0.12). In a separate analysis, the effect of the Ala222Val polymorphism in the MTHFR gene on the association of homocysteine level with fracture risk was studied. No effect of presence of the 222Val allele on the risk estimates for fractures were observed.

### 3. Conclusions

There was no significant relation between the MTHFR 222-Val variant and serum homocysteine levels in the study population. There was no effect of the 222-Val allele on the association of homocysteine level with fracture risk.

While the preferred embodiment of the invention has been illustrated and described, it will be appreciated that various changes can be made therein without departing from the spirit and scope of the invention.

1-32. (canceled)

33. A method of determining the susceptibility of a human subject to non-vertebral fractures, comprising determining which allele(s) of polymorphisms in methyltetrahydrofolate reductase (MTHFR) and collagen Iα1 are present in the subject, wherein the presence of the C677T MTHFR polymorphism and a COLIA1 polymorphism are indicative of an increased susceptibility of non-vertebral fractures.

34. The method of claim 33, wherein the alleles are determined by amplification of a relevant portion of the MTHFR and collagen Iα1 genes.

35. The method of claim 33, wherein said method is performed in vitro.

36. The method of claim 35, wherein said method is performed on blood or tissue samples of said subject.

37. The method of claim 33, wherein the subject is a female.

38. A method for preventing or reducing the susceptibility of non-vertebral fracture in a human subject, comprising the steps of:
(a) determining that the subject has an increased susceptibility to non-vertebral fracture, wherein the presence of a C677T MTHFR polymorphism and a COLIA1 polymorphism are indicative of an increased susceptibility to non-vertebral fracture; and
(b) prescribing or administering folic acid or folate to the subject when said polymorphisms are present to prevent or reduce said susceptibility.

39. A method for determining the susceptibility of a living human subject to non vertebral fracture, comprising using a kit comprising one or more nucleic acid primer molecules for amplification of a portion of the MTHFR and the collagen Iα1 gene from said subject to determine whether the C677T MTHFR polymorphism and a COLIA1 polymorphism are present, and determining whether said polymorphisms are present in said subject; wherein the presence of the C677T MTHFR polymorphism and the COLIA1 polymorphism are indicative of an increased susceptibility of non-vertebral fracture.

40. The method of claim 38, wherein the subject is a female.

41. The method of claim 39, wherein the subject is a female.

42. The method of claim 33, wherein the increased susceptibility of non-vertebral fractures is independent of the bone mineral density of the subject.

43. The method of claim 38, wherein the increased susceptibility of non-vertebral fractures is independent of the bone mineral density of the subject.

44. The method of claim 39, wherein the increased susceptibility of non-vertebral vertebral fractures is independent of the bone mineral density of the subject.
